SEQUENCE LISTING (22 sequences)

SEQ ID NO: 1 - DNA, 21 bp, Artificial Sequence
  Synthetic forward primer for Cdc42
ctgccaaccatgacaacctaa

SEQ ID NO: 2 - DNA, 21 bp, Artificial Sequence
  Synthetic reverse primer for Cdc42
agacaaaacaacaaggtccag

SEQ ID NO: 3 - DNA, 51 bp, Artificial Sequence
  Synthetic polynucleotide of loxp1
gacacggatccataacttcgtataatgtatgctatacgaagttatcgagtc

SEQ ID NO: 4 - DNA, 52 bp, Artificial Sequence
  Synthetic polynucleotide of loxp2
ccgcggataacttcgtataatgtatgctatacgaagttatactagtccaacg

SEQ ID NO: 5 - DNA, 271 bp, Artificial Sequence
  Synthetic tetracycline response element (TRE)
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagaga

SEQ ID NO: 6 - DNA, 39 bp, Artificial Sequence
  Synthetic CMV promoter
ggtaggcgtgtacggtgggaggcctatataagcagagct

SEQ ID NO: 7 - DNA, 747 bp, Mus musculus
atgagaactccgctgctaccgctggcgcgctcagtgctgttgctgctggtcttaggctcaggccattatgcagctgctttggagctcaatgaccccagctcagggaaaggcgaatcgctttctggggaccacagtgccggtggacttgagctttctgtgggaagagaggtttccaccataagcgaaatgccttctggcagtgaactctccacaggggactacgactactcagaggagtatgataatgaaccacaaatatccggctatattatagatgattcagtcagagttgaacaggtgattaagcccaagaaaaacaagacagaaggagaaaagtctacagaaaaacccaaaaggaagaaaaagggaggcaaaaatggaaaaggcagaaggaataagaagaaaaagaatccatgcactgccaagtttcagaacttttgcattcatggcgaatgcagatacatcgagaacctggaggtggtgacatgcaattgtcatcaagattactttggtgaacggtgtggagaaaaatccatgaagactcacagcgaggatgacaaggacctatccaagattgcagtagtagctgtcactatctttgtctctgccatcatcctcgcagctattggcatcggcatcgttatcacagtgcacctttggaaacgatacttcagggaatatgaaggagaaacagaagaaagaaggaggcttcgacaagaaaacgggactgtgcatgccattgcctag

SEQ ID NO: 8 - DNA, 23 bp, Artificial Sequence
  Synthetic forward primer for Gapdh
aaggtcggtgtgaacggatttgg

SEQ ID NO: 9 - DNA, 23 bp, Artificial Sequence
  Synthetic reverse primer for Gapdh
cgttgaatttgccgtgagtggag

SEQ ID NO: 10 - DNA, 23 bp, Artificial Sequence
  Synthetic forward primer for Areg
gcagatacatcgagaacctggag

SEQ ID NO: 11 - DNA, 22 bp, Artificial Sequence
  Synthetic reverse primer for Areg
ccttgtcatcctcgctgtgagt

SEQ ID NO: 12 - DNA, 22 bp, Artificial Sequence
  Synthetic forward primer for Col1a1
cctcagggtattgctggacaac

SEQ ID NO: 13 - DNA, 22 bp, Artificial Sequence
  Synthetic reverse primer for Col1a1
cagaaggaccttgtttgccagg

SEQ ID NO: 14 - DNA, 23 bp, Artificial Sequence
  Synthetic forward primer for Areg
aaacaaaacaagctgaaatgtgg

SEQ ID NO: 15 - DNA, 22 bp, Artificial Sequence
  Synthetic reverse primer for Areg
aaggcctttaagaacaagttgt

SEQ ID NO: 16 - DNA, 1128 bp, Mus musculus
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatgttcgaacgaagttcctattctctagaaagtataggaacttcgctagactagtacgcgtgtacaccttgtaattgctgctctgagcaagttgccattttttctttttagaggttttcagtcatagcagtaatgctagttctggtttgagtggctgagcctgttgctaggggaaaaaagtatggatttaaacataaatcaataaaataattgtctttaatttcttcttaggacaagatctaatttgaaatattaaaagtggatacaaaactgtttccgaaatgcagacaattaagtgtgttgttgttggtgatggtgctgttggtaaaacatgtctcctgatatcctacacaacaaacaaattcccatcggaatatgtaccaactgtaagtataaaggctttttactagcaaaagattgtaatgtagtgtctgtccattggaaaacacttggcctgcctgcagtatttttgactgtcttgccctttaaaaaaaattaaattttactacctttattactttgtggggtgtgtgttataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 17 - DNA, 561 bp, Mus musculus
tgttctattttaaagtacaggtaatcatgcatgagaagtcaaaacctttaaaactgtcaaacagtgggctgctgtgtgtggcatttgctgccaaccatgacaacctaagttcaacttaagagcccaacaatggaaaaagaccccttcaagttgtcctctgccatctacacatacaccaaagcaggacacaggtatgtacagaattcataacttcgtataatgtatgctatacgaagttatggtaccgaattcagtttctggaccttgttgttttgtcttaagtatcaaagtagaacagtgaccgatatattccttttatttttttttttcttccctgagactgggtttctctgtgtagcccttgctgttctgtaactcactctgtgagtggcctcaaactcagagatccgcctgccttgggcaaggaaggtgctataaaaagagtctcgtgtggtatatgaagtatagtttgtgaaagctgcttcagtgtgagcacacacgcattatatgcaagaccaattgcagcccgaagaatactctaaaaaatgactcactgcccag

SEQ ID NO: 18 - DNA, 1204 bp, Mus musculus
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccgggatgagaactccgctgctaccgctggcgcgctcagtgctgttgctgctggtcttaggctcaggccattatgcagctgctttggagctcaatgaccccagctcagggaaaggcgaatcgctttctggggaccacagtgccggtggacttgagctttctgtgggaagagaggtttccaccataagcgaaatgccttctggcagtgaactctccacaggggactacgactactcagaggagtatgataatgaaccacaaatatccggctatattatagatgattcagtcagagttgaacaggtgattaagcccaagaaaaacaagacagaaggagaaaagtctacagaaaaacccaaaaggaagaaaaagggaggcaaaaatggaaaaggcagaaggaataagaagaaaaagaatccatgcactgccaagtttcagaacttttgcattcatggcgaatgcagatacatcgagaacctggaggtggtgacatgcaattgtcatcaagattactttggtgaacggtgtggagaaaaatccatgaagactcacagcgaggatgacaaggacctatccaagattgcagtagtagctgtcactatctttgtctctgccatcatcctcgcagctattggcatcggcatcgttatcacagtgcacctttggaaacgatacttcagggaatatgaaggagaaacagaagaaagaaggaggcttcgacaagaaaacgggactgtgcatgccattgcctag

SEQ ID NO: 19 - DNA, 21 bp, Artificial Sequence
  Synthetic forward primer for teto-Areg
gtacccgggatgagaactccg

SEQ ID NO: 20 - DNA, 21 bp, Artificial Sequence
  Synthetic reverse primer for teto-Areg
gccggatatttgtggttcatt

SEQ ID NO: 21 - DNA, 987 bp, Mus musculus
ctctatgtcatgcgaggtccagtacatctaactacaggcatacctaggtaaaagaattcaagtggcatgcaacagaggttactgtgcactgcccgatagaggacacggatccataacttcgtataatgtatgctatacgaagttatcgagtcgctattctgtgcagctttatcatgtgtgtgttgctttaattaatgaggccaaagtagctctcagtgctgtgatttctgggtctcacgtaacccaaatcattttgaagacgaaaagagaacgtgggcagtcgtaaatctaatcttactttgtcaaacttcctttctctccagttgaacaggtgattaagcccaagaaaaacaagacagaaggagaaaagtctacagaaaaacccaaaaggaagaaaaagggaggcaaaaatggaaaaggcagaaggaataagaagaaaaagaatccatgcactgccaagtttcagaacttttgcattcatggcgaatgcagatacatcgagaacctggaggtggtgacatgcaagtaggtttgtttcctacacaacacctgaaatccccatcaatagaaactattcacttttccagtgtgtaaaccaaggatttcatgagccaacattatgtttgtacaggcaattaaaatataagcatgtaaatcccgcggataacttcgtataatgtatgctatacgaagttatactagtccaacggaaaaaagattcttagcttaaaggctgtaacaaatagctttatggctactggtgcacagtatcattttattataaatattatatgtatgcaatgtatatatatatgtgcctgtacatatattttaagccttaaaaaaaacttaaagtatttatcacacaacttttcatgttgttctaatgtccccaaaccctctcaacgcactaaaactaaactaaatgatagaggaatgtattagctgtgacaccaggagtcaaagtcatcgcttggtcctaaaaga

SEQ ID NO: 22 - DNA, 430 bp, Mus musculus
ctctatgtcatgcgaggtccagtacatctaactacaggcatacctaggtaaaagaattcaagtggcatgcaacagaggttactgtgcactgcccgatagaggacacggatccataacttcgtataatgtatgctatacgaagttatcgagtcgaaaaaagattcttagcttaaaggctgtaacaaatagctttatggctactggtgcacagtatcattttattataaatattatatgtatgcaatgtatatatatatgtgcctgtacatatattttaagccttaaaaaaaacttaaagtatttatcacacaacttttcatgttgttctaatgtccccaaaccctctcaacgcactaaaactaaactaaatgatagaggaatgtattagctgtgacaccaggagtcaaagtcatcgcttggtcctaaaaga

(19) United States
(12) Patent Application Publication
(10) Pub. No.: US 2022/0275055 A1
(43) Pub. Date: Sep. 1, 2022

(54) DRUG TARGET OF IDIOPATHIC PULMONARY FIBROSIS

(71) Applicant: National Institute of Biological Sciences, Beijing, Beijing (CN)

(72) Inventors: Nan Tang, Beijing (CN); Huijuan Wu, Beijing (CN)

(73) Assignee: National Institute of Biological Sciences, Beijing, Beijing (CN)

(21) Appl. No.: 17/614,673

(22) PCT Filed: May 30, 2019
(86) PCT No.: PCT/CN2019/089358

§ 371 Date: Nov. 29, 2021

(51) Int. Cl. C07K 14/71; A01K 67/027; G01N 33/68

(52) U.S. Cl. CPC C07K 14/71; A01K 67/0276; G01N 33/6893; G01N 2800/12; A01K 2217/075; A01K 2227/105; A01K 2267/035

(57) ABSTRACT

Provided is a drug target for idiopathic pulmonary fibrosis, and the use thereof. The drug target is AREG signaling in AT2 cells of the lung. The drug target can be used to screen drugs for treating and/or preventing pulmonary fibrosis, in particular, idiopathic pulmonary fibrosis (IPF) of animals and human beings.

### PRIORITY CLAIM AND CROSS-REFERENCE

The present application is a National Stage Application, filed under 35 U.S.C. 371, of International Patent Application No. PCT/CN2019/089358, filed on May 30, 2019, which is incorporated herein by reference in its entirety.

### SEQUENCE LISTING

This application contains a Sequence Listing, which is submitted electronically via EFS-Web in ASCII format with a file name H5292-00001-SEQTXT, creation date of Mar. 3, 2022, and a size of 11 kB. This sequence listing submitted is part of the specification and is herein incorporated by reference in its entirety.

### Introduction

Fibrosis, the thickening and scarring of connective tissue that can result from injury, is characterized by the excessive proliferation of fibroblast cells and the accumulation of extracellular matrix (ECM) components. This disorder, which is commonly observed in organs including lungs, livers, and kidneys, among many others, causes disrupted tissue architecture and leads to major impairments in organ function^{1,2}. Indeed, fibrosis can develop in nearly every organ and is a major cause of end-stage organ failure and death in a large variety of chronic diseases³. A common feature of pulmonary fibrosis is the excessive proliferation of fibroblasts around the air sacs of lungs (alveoli)⁴. Extensive biomedical studies have established that an increased number of fibroblasts, in combination with their excessive ECM deposition in the lung ultimately cause alveolar structure destruction, decreased lung compliance, and disrupted gas exchange function⁵⁻⁷.

The most common type of pulmonary fibrosis is idiopathic pulmonary fibrosis (IPF). This disorder eventually affects entire lung lobes, but it begins with microscopic fibrotic lesions that occur at the peripheral regions and slowly progress inward, and this fibrosis can ultimately lead to respiratory failure^{8,9}. IPF is a fatal disease with the median survival time of only 2-4 years from diagnosis¹⁰. Scientifically, the mechanisms and nature of the pathological progression of IPF are not fully understood, although multiple studies have implicated contributions from a specific subset of alveolar epithelial cells-alveolar type II (AT2) cells^{4,11}.

The pulmonary fibrosis patient has decreased lung compliance, disrupted gas exchange, and ultimately respiratory failure and death. It is estimated that IPF affects 1 of 200 adults over the age of 65 in the United States, with a median survival time of 2-4 years. In China, the estimated incidence of IPF is 3-5/100,000, accounting for about 65% of all interstitial lung diseases. The diagnosis is usually made between 50 and 70 years old, and the ratio of male to female is 1.5 to 2:1. The survival time of the patient is usually only 2-5 years.

Currently, there is no cure for IPF. Two known drugs, nintedanib and pirfenidone, have similar effects on the rate of decline in forced vital capacity over 1 year. Although the both drugs showed a tendency of reducing mortality, these two drugs failed to show significantly increased survival time. One of main reasons is that there is no ideal drug target of pulmonary fibrosis, in particular, idiopathic pulmonary fibrosis (IPF), so as to screen candidate drugs for treating pulmonary fibrosis, in particular, idiopathic pulmonary fibrosis (IPF).

### Summary of the Invention

The present invention relates to a drug target for idiopathic pulmonary fibrosis, and the use thereof. The drug target is AREG signaling in AT2 cells of the lung. The drug target can be used to screen drugs for treating and/or preventing pulmonary fibrosis, in particular, idiopathic pulmonary fibrosis (IPF) of animals and human beings. The present invention further provides a method for screening candidate drugs for treating pulmonary fibrosis, in particular, idiopathic pulmonary fibrosis (IPF) of animals and human beings using the drug target.

In the first place, the present invention provides a drug target for idiopathic pulmonary fibrosis. The drug target is AREG signaling in AT2 cells of the lung, which refers to AREG target hereafter.

It is found in the present invention that AREG was detected in AT2 cells of all IPF specimens but was not detected in AT2 cells of control lungs.

It is found in the present invention that no AREG signal can be detected in a control lung of a subject with or without PNX. No AREG signal can be detected in AT2 cells of a control lung from a subject with or without PNX.

It is further found in the present invention that AREG can be detected in AT2 cells of Cdc42 AT2 null lungs. The expression levels of AREG are gradually increased in the lungs of Cdc42 AT2 null lungs after PNX.

Therefore, the expression level of AREG is significantly up-regulated in AT2 cells of both progressive fibrosis mouse model and lung fibrosis patients.

It is further in the present invention found that overexpression of AREG in AT2 cells is sufficiently to induce lung fibrosis.

Preferably, ectopic expression of AREG in AT2 cells is sufficiently to induce lung fibrosis.

Preferably, the AREG target is AREG in AT2 cells of lung from a subject.

Preferably, the AREG target is a receptor of AREG in AT2 cells of lung from a subject.

Preferably, the AREG target is EGFR in fibroblasts of lung from a subject.

The present invention demonstrates that the strength of EGFR signaling in α-SMA positive fibroblasts is dependent on the AREG expression in AT2 cells.

The present invention demonstrates that reducing the expression levels of AREG in AT2 cells of lungs from a subject significantly attenuates the development of pulmonary fibrosis of Cdc42 AT2 null mice.

Therefore, the present invention indicates that AREG, and its receptor, EGFR are therapeutic targets for treating fibrosis.

In the second place, the present invention provides a method for generating Areg AT2 overexpression transgenic mice, wherein AREG is specifically overexpressed in lung AT2 cells.

Preferably, the said method involves a step of specifically inducing the expression of Areg in AT2 cells after the doxycycline treatment. Preferably, the generated transgenic mouse is Spc-rtTA; teto-Areg mouse. Preferably, the Spc-rtTA; teto-Areg mouse has a chacterized sequence shown by SEQ ID NO:18.

Preferably, the Spc-rtTA; teto-Areg mouse may be identified using the following primer sequences:

Forward:
(SEQ ID NO: 19)
GTACCCGGGATGAGAACTCCG;
Reverse:
(SEQ ID NO: 20)
GCCGGATATTTGTGGTTCATT.

In the third place, the present invention provides a transgenic mouse, wherein AREG is specifically overexpressed in AT2 cells of lungs. The mouse is an Areg AT2 overexpression transgenic mouse.

Preferably, in the transgenic mouse, the expression of Areg was induced specifically in AT2 cells after the doxycycline treatment. Preferably, the transgenic mouse is Spc-rtTA; teto-Areg mouse. Preferably, the Spc-rtTA; teto-Areg mouse has a chacterized sequence shown by SEQ ID NO:18.

Preferably, the Spc-rtTA; teto-Areg mouse may be identified using the following primer sequences:

Forward:
(SEQ ID NO: 19)
GTACCCGGGATGAGAACTCCG;
Reverse:
(SEQ ID NO: 20)
GCCGGATATTTGTGGTTCATT.

In the fourth place, the present invention provides use of AREG in AT2 cells and/or its receptor EGFR in fibroblasts of lungs as a drug target for treating pulmonary fibrosis, in particular, idiopathic pulmonary fibrosis (IPF) of animals and human beings.

In the fifth place, the present invention provides use of AREG target or the above transgenic mouse for screening a drug for treating pulmonary fibrosis, in particular, idiopathic pulmonary fibrosis (IPF) of animals and human beings.

In the sixth place, the present invention provides use of a detector of AREG and/or a detector of its receptor EGFR in manufacturing a diagnosis kit for diagnosing pulmonary fibrosis, in particular, idiopathic pulmonary fibrosis (IPF) of animals and human beings.

Preferably, the kit may be used to the sample from the subject suspecting suffering pulmonary fibrosis, in particular, idiopathic pulmonary fibrosis (IPF). The sample may be the biopsy tissue. For example, the biopsy tissue may be lung tissue from the subject. Preferably, the biopsy tissue may be the lower part, the middle part or the upper part of the lung lobe from a subject. If AREG may be detected in the upper part of the lung lobe from a subject, the subject may be diagnosed as suffering a severe pulmonary fibrosis, in particular, idiopathic pulmonary fibrosis (IPF). The most common type of lung fibrosis is known as idiopathic pulmonary fibrosis, in which fibrotic lesions start at the periphery of the lung lobe, and progress towards the center of the lung lobe, then the upper side of the lung lobe, and eventually causing respiratory failure.

In the seventh place, the present invention provides use of substance targeting AREG in AT2 cells and/or its receptor, for example, EGFR in fibroblasts of lungs in manufacturing a medicament for treating pulmonary fibrosis, in particular, idiopathic pulmonary fibrosis (IPF) of animals and human beings.

Preferably, the substance is an inhibitor of AREG in AT2 cells, or is an inhibitor of EGFR in fibroblasts of lungs.

The animal may be mouse, rabbit, rat, canine, pig, horse, cow, sheep, monkey or chimpanzee.

The invention encompasses all combination of the particular embodiments recited herein.

### BRIEF DESCRIPTION OF THE DRAWINGS

FIG. 1 shows generating a mouse line in which Cdc4 2 gene is specifically deleted in AT2 cells.

FIG. 2 shows the fragments of Cdc42 DNA sequence before and after deleting the exon2 of the Cdc42 gene in AT2 cells.

FIG. 3 shows that loss of Cdc42 gene in AT2 cells impairs the differentiation of AT2 cells during either post-PNX alveolar regeneration or alveolar homeostasis.

FIG. 4 shows that loss of Cdc42 in AT2 cells leads to progressive lung fibrosis in PNX-treated mice.

FIG. 5 shows that loss of Cdc42 in AT2 cells leads to progressive lung fibrosis in non-PNX-treated aged mice.

FIG. 6 shows the development of α-SMA⁺ fibroblastic foci in the lungs of Cdc42 AT2 null mice.

FIG. 7 shows that AREG is strongly and specifically expressed in AT2 cells of Cdc42 AT2 null lungs.

FIG. 8 shows that AREG is strongly and specifically expressed in AT2 cells of human pulmonary fibrosis patients.

FIG. 9 shows that the sequence of teto-Areg.

FIG. 10 shows that the expression of Areg is induced specifically in AT2 cells of Spc-rtTA; teto-Areg mice after the doxycycline treatment. Overexpressing AREG in AT2 cells is sufficiently to induce lung fibrosis.

FIG. 11 shows the fragments of Areg DNA sequence before and after deleting the exon3 of the Areg gene in AT2 cells.

FIG. 12 shows that deletion of Areg gene in AT2 cells of Cdc42 AT2 null lungs significantly attenuated the development of lung fibrosis.

FIG. 13 shows targeting AREG and its receptor, EGFR, so as to treat IPF and other fibrosis diseases.

### DESCRIPTION OF PARTICULAR EMBODIMENTS OF THE INVENTION

The descriptions of particular embodiments and examples are provided by way of illustration and not by way of limitation. Those skilled in the art will readily recognize a variety of noncritical parameters that could be changed or modified to yield essentially similar results.

The idiopathic pulmonary fibrosis (IPF) is a type of chronic lung disease characterized by a progressive and irreversible decline in lung function. Symptoms typically include gradual onset of shortness of breath and a dry cough. Other changes may include feeling tired and nail clubbing. Complications may include pulmonary hypertension, heart failure, pneumonia, or pulmonary embolism.

The alveolar epithelia of lungs are composed of a combination of both alveolar type I (AT1) and type II (AT2) cells. AT2 cells are the alveolar stem cells, and can differentiate into AT1 cells during alveolar homeostasis and post-injury repair^{12,13}. AT1 cells-which ultimately comprise fully 95% of the alveolar surface in adult lungs-are large squamous cells that function as the epithelial component of the thin air-blood barrier¹⁴. In IPF tissues, abnormal hyperplastic AT2 cells are typically located adjacent to fibroblastic foci¹⁵, and the gene mutants that affect the functions of AT2 cells are frequently observed in IPF tissues in the clinic^{16,17}. In addition, recent advances in identifying the molecular profiles of IPF lungs showed that TGFβ signaling (a common fibrotic signaling in many fibrotic diseases) is activated in the AT2 cells of IPF lungs¹⁸. These multiple lines of evidence collectively demonstrate an obvious pathological impact of AT2 cells in lung fibrosis, yet the precise pathological mechanisms underlying abnormal AT2 physiology and progressive pulmonary fibrosis remain to be elucidated.

The Sftpc gene promoter-driven recombinase (Spc-CreER) is used to specifically delete genes in AT2 cells after administration of tamoxifen to the animal. The CreER mouse system is commonly used for inducible gene knockout studies.

Amphiregulin (AREG) is a member of the epidermal growth factor family. AREG is synthesized as a membrane-anchored precursor protein, which can directly function on adjacent cells as a juxtacrine factor. After proteolytic processing by cell membrane proteases (TACE/ADAM17), AREG is secreted and functions as an autocrine or paracrine factor. AREG is a ligand of the epidermal growth factor receptor (EGFR), a transmembrane tyrosine kinase. By binding to EGFR, AREG can activate major intracellular signaling cascades that control cell survival, proliferation, and differentiation¹⁹⁻²¹.

Physiologically, AREG plays an important role in the development and maturation of mammary glands, bone tissue, and oocytes^{20,22}. At normal conditions, AREG is expressed in low levels in adult tissues, except placenta. However, the chronic elevation of AREG expression has been shown to be associated with some pathological conditions. The increased expression of AREG is associated with a psoriasis-like skin phenotype and some inflammatory conditions²³. Several studies have described the oncogenic activity of AREG in lung, breast, colorectal, ovary and prostate carcinomas, as well as in some hematological and mesenchymal cancers^{24,25}. In addition, AREG may be involved in resistance to several cancer treatments^{26,27}.

It has been shown that TGFβ can activate the expression of AREG in bleomycin-induced lung fibrosis mouse model²⁸. It was shown that the expression level of AREG increases in liver fibrosis, cystic fibrosis, and polycystic kidney disease²³. It is therefore hypothesized that AREG may contribute to the growth and survival of fibrogenic cells during these fibrotic disease, especial idiopathic pulmonary fibrosis(IPF). However, scientifically, the mechanisms and nature of the pathological progression of IPF are not fully understood²⁹. Although it was speculated that AREG might play a function in IPF development, the cell that express AREG during progressive lung fibrosis remains unknown. In addition, the effect of targeting AREG in progressive lung fibrosis is unknown due to lack of a progressive lung fibrosis mouse model.

In an embodiment of the present invention, it is shown that no AREG signal can be detected in a control lung of a subject with or without PNX, and further, no AREG signal can be detected in AT2 cells of a control lung from a subject with or without PNX.

In an embodiment of the present invention, it is shown that AREG can be detected in AT2 cells of PNX-treated Cdc42 AT2 null lungs or aged Cdc42 AT2 null mice, the expression levels of AREG are gradually increased in the lungs of Cdc42 AT2 null lungs after PNX, and remarkably, AREG was detected in AT2 cells of all IPF specimens. Therefore, the present invention first shows that the expression level of AREG is significantly up-regulated in AT2 cells of the both progressive fibrosis mouse model and lung fibrosis patients.

In an embodiment of the present invention, a transgenic mouse, wherein AREG is specifically overexpressed in AT2 cells of the lung, is generated. The transgenic mouse has obvious fibrotic changes in the lung.

In an embodiment of the present invention, a transgenic mouse, wherein both Areg gene and Cdc42 gene are null, is generated. This transgenic mouse is an Areg&Cdc42 AT2 double null mouse. Lungs of Areg&Cdc42 AT2 double null mice showed minimal fibrosis at post-PNX day 21, as compared to the significant lung fibrosis in Cdc42 AT2 null lungs. Therefore, reducing the expression levels of AREG significantly attenuated the development of pulmonary fibrosis of Cdc42 AT2 null mice. Accordingly, the present invention suggests that AREG and its receptor, EGFR, are therapeutic targets for treating fibrosis. AREG means AREG in AT2 cells of lung, and EGFR means EGFR on the fibroblasts of lungs.

In an embodiment of the present invention, it is shown that blocking AREG and its receptor, EGFR, can be a therapeutic approach for treating the IPF and other fibrosis diseases.

### EXAMPLES

Methods

Mice and Survival Curve Record

Rosa26-CAG-mTmG (Rosa26-mTmG), and Cdc42^{flox/flox} mice³⁰ have been described previously. All experiments were performed in accordance with the recommendations in the Guide for Care and Use of Laboratory Animals of the National Institute of Biological Sciences. To monitor the survival of mice, both the Control and the Cdc42AT2 null mice were weighed every week after the PNX treatment. Once the mice reached the pre-defined criteria for end-points, the mice were sacrificed. We define the endpoints according to the pre-defined criteria^{31,32}.

Generating Spc-CreER;rtTA (Spc-CreER) knock-in mice. The CreERT2, p2a, and rtTA element were enzyme-linked and inserted into the mouse endogenous Sftpc gene. The insertion site is the stop codon of the endogenous Sftpc gene, then a new stop codon was created at the 3′ end of rtTA. The CRISPR/Cas9 technology was used to insert the CreERT2-p2a-rtTA fragment into the genome.

Generating Areg^{flox/flox} Mice

The Areg^{flox/flox} mice were generated according to the previous work³³. Briefly, the Areg exon3 was anchored by loxp. The loxp1 (GACACGGATCCATAACTTCGTATAATGTATGCTATACGAAGTTATCGAGTC (SEQ ID NO:3)) was inserted into the Areg DNA position 3704, and the loxp2 (CCGCGGATAACTTCGTATAATGTATGCTATACGAAGTTATACTAGTCCAACG(SEQ ID NO:4)) was inserted into the Areg DNA position 4208. After the tamoxifen-induced Cre-loxP recombination, the exon3 of Areg gene was deleted, and then the AREG function was blocked.

Generating Teto-Areg Mice

Inserting a tetracycline response element before CMV promoter-driven Areg so that the expression of Areg can induced when mice are treated with doxycycline (Dox). The sequence of tetracycline response element is shown as followed:

(SEQ ID NO: 5)
5′TCCCTATCAGTGATAGAGAAAAGTGAAAGTCGAGTTTACCACTCCCTA
TCAGTGATAGAGAAAAGTGAAAGTCGAGTTTACCACTCCCTATCAGTGAT
AGAGAAAAGTGAAAGTCGAGTTTACCACTCCCTATCAGTGATAGAGAAAA
GTGAAAGTCGAGTTTACCACTCCCTATCAGTGATAGAGAAAAGTGAAAGT
CGAGTTTACCACTCCCTATCAGTGATAGAGAAAAGTGAAAGTCGAGTTTA
CCACTCCCTATCAGTGATAGAGA3′.

Inserting a minimal CMV promoter before Areg CDNA so that Areg is overexpressed. The sequence of CMV promter is shown as followed:

(SEQ ID NO: 6)
5′GGTAGGCGTGTACGGTGGGAGGCCTATATAAGCAGAGCT3′.

The sequence of Areg cDNA is shown as followed:

(SEQ ID NO: 7)
5′ATGAGAACTCCGCTGCTACCGCTGGCGCGCTCAGTGCTGTTGCTGCTG
GTCTTAGGCTCAGGCCATTATGCAGCTGCTTTGGAGCTCAATGACCCCAG
CTCAGGGAAAGGCGAATCGCTTTCTGGGGACCACAGTGCCGGTGGACTTG
AGCTTTCTGTGGGAAGAGAGGTTTCCACCATAAGCGAAATGCCTTCTGGC
AGTGAACTCTCCACAGGGGACTACGACTACTCAGAGGAGTATGATAATGA
ACCACAAATATCCGGCTATATTATAGATGATTCAGTCAGAGTTGAACAGG
TGATTAAGCCCAAGAAAAACAAGACAGAAGGAGAAAAGTCTACAGAAAAA
CCCAAAAGGAAGAAAAAGGGAGGCAAAAATGGAAAAGGCAGAAGGAATAA
GAAGAAAAAGAATCCATGCACTGCCAAGTTTCAGAACTTTTGCATTCATG
GCGAATGCAGATACATCGAGAACCTGGAGGTGGTGACATGCAATTGTCAT
CAAGATTACTTTGGTGAACGGTGTGGAGAAAAATCCATGAAGACTCACAG
CGAGGATGACAAGGACCTATCCAAGATTGCAGTAGTAGCTGTCACTATCT
TTGTCTCTGCCATCATCCTCGCAGCTATTGGCATCGGCATCGTTATCACA
GTGCACCTTTGGAAACGATACTTCAGGGAATATGAAGGAGAAACAGAAGA
AAGAAGGAGGCTTCGACAAGAAAACGGGACTGTGCATGCCATTGCCTAG
3′.

The tetracycline response element, CMV promoter, and Areg CDNA were enzyme-linked and inserted into the mouse genome. The sequence of teto-Areg is shown as followed:

(SEQ ID NO: 18)
5′TCCCTATCAGTGATAGAGAAAAGTGAAAGTCGAGTTTACCACTCCCTA
TCAGTGATAGAGAAAAGTGAAAGTCGAGTTTACCACTCCCTATCAGTGAT
AGAGAAAAGTGAAAGTCGAGTTTACCACTCCCTATCAGTGATAGAGAAAA
GTGAAAGTCGAGTTTACCACTCCCTATCAGTGATAGAGAAAAGTGAAAGT
CGAGTTTACCACTCCCTATCAGTGATAGAGAAAAGTGAAAGTCGAGTTTA
CCACTCCCTATCAGTGATAGAGAAAAGTGAAAGTCGAGCTCGGTACCCGG
GTCGAGGTAGGCGTGTACGGTGGGAGGCCTATATAAGCAGAGCTCGTTTA
GTGAACCGTCAGATCGCCTGGAGACGCCATCCACGCTGTTTTGACCTCCA
TAGAAGACACCGGGACCGATCCAGCCTCCGCGGCCCCGAATTCGAGCTCG
GTACCCGGGATGAGAACTCCGCTGCTACCGCTGGCGCGCTCAGTGCTGTT
GCTGCTGGTCTTAGGCTCAGGCCATTATGCAGCTGCTTTGGAGCTCAATG
ACCCCAGCTCAGGGAAAGGCGAATCGCTTTCTGGGGACCACAGTGCCGGT
GGACTTGAGCTTTCTGTGGGAAGAGAGGTTTCCACCATAAGCGAAATGCC
TTCTGGCAGTGAACTCTCCACAGGGGACTACGACTACTCAGAGGAGTATG
ATAATGAACCACAAATATCCGGCTATATTATAGATGATTCAGTCAGAGTT
GAACAGGTGATTAAGCCCAAGAAAAACAAGACAGAAGGAGAAAAGTCTAC
AGAAAAACCCAAAAGGAAGAAAAAGGGAGGCAAAAATGGAAAAGGCAGAA
GGAATAAGAAGAAAAAGAATCCATGCACTGCCAAGTTTCAGAACTTTTGC
ATTCATGGCGAATGCAGATACATCGAGAACCTGGAGGTGGTGACATGCAA
TTGTCATCAAGATTACTTTGGTGAACGGTGTGGAGAAAAATCCATGAAGA
CTCACAGCGAGGATGACAAGGACCTATCCAAGATTGCAGTAGTAGCTGTC
ACTATCTTTGTCTCTGCCATCATCCTCGCAGCTATTGGCATCGGCATCGT
TATCACAGTGCACCTTTGGAAACGATACTTCAGGGAATATGAAGGAGAAA
CAGAAGAAAGAAGGAGGCTTCGACAAGAAAACGGGACTGTGCATGCCATT
GCCTAG3′.

In Spc-rtTA; teto-Areg mice, the expression of Areg was induced specifically in AT2 cells after the doxycycline treatment.

Primer sequences for
sequencing teto-Areg sequence:
Forward:
(SEQ ID NO: 19)
GTACCCGGGATGAGAACTCCG;
Reverse:
(SEQ ID NO: 20)
GCCGGATATTTGTGGTTCATT.

Pneumonectomy (PNX)

The male mice of 8 weeks old were injected with tamoxifen (dosage: 75mg/kg) every other day for 4 times. The mice were anesthetized and connected to a ventilator (Kent Scientific, Topo) from 14th day after the final dose of tamoxifen injection. The chest wall was incised at the fourth intercostal ribs and the left lung lobe was removed.

Pulmonary Function Test

Lung function parameters were measured using the invasive pulmonary function testing system (DSI Buxco® PFT Controller). Mice were first anesthetized before inserting an endotracheal cannula into their trachea. The dynamic compliance results were obtained from the Resistance & Compliance Test. The forced vital capacity results were obtained from the Pressure Volume Test.

Hematoxylin and Eosin (H&E) Staining and Immunostaining

Lungs were inflated with 4% paraformaldehyde (PFA) and were continually fixed in 4% PFA at 4° C. for 24 hours. Then the lungs were cryoprotected in 30% sucrose and embedded in OCT (Tissue Tek).

The H&E staining experiment followed the standard H&E protocol. Briefly, slides were washed by water to remove the OCT. The nuclei were stained by hemotoxylin (Abcam, ab150678) for 2 minutes and the cytoplasm were stained by eosin (Sigma, HT110280) for 3 minutes. Slices were sealed with neutral resin after the dehydration and clearing steps.

The immunofluorescence staining experiments followed the protocol previously described³⁴. In brief, after removing the OCT, the lung slices were blocked with 3%BSA/0.1%TritonX-100/PBS for 1 hour, and then slides were incubated with primary antibodies at 4° C. for overnight. After washing the slides with 0.1%TritonX-100/PBS for 3 times, the slices were incubated with secondary antibodies for 2 hours at room temperature.

The primary antibodies used herein are listed below:

| Name | Company and catalog number | Dilution |
|---|---|---|
| Chicken anti-GFP | Abcam, ab13970-100 | 1:500 |
| Rabbit anti-Collagen I | Abcam, ab34710 | 1:300 |
| Mouse anti α-SMA | Sigma, C6198 | 1:300 |
| Rat anti-Ki67 | Bioscience, 514-5698-82 | 1:300 |
| Rabbit anti-Prospc | Millipore, ab3786 | 1:500 |
| Goat anti-Prospc | Santa Cruz, sc-7706 | 1:200 |
| Rabbit anti pSmad2 | CST, #3101 | 1:500 |
| Mouse anti HT2-280 | Terrace Biotech, TB-27AHT2-280 | 1:50 |
| Hamster anti-Pdpn | Developmental Studies | 1:100 |
| | Hybridoma Bank, clone8.1.1 | |
| Anti-AREG | Bioss, bs-3847r | 1:100 |

The secondary antibodies used herein are listed below:

| Name | Company and catalog number | Dilution |
|---|---|---|
| Alexa Fluor 488 Donkey | 703-545-155, Jackson | 1:500 |
| anti-Chicken | Immuno Research | |
| Alexa Fluor 488 Donkey | 715-545-150, Jackson | 1:500 |
| anti-mouse | Immuno Research | |
| Alexa Fluor 568 Donkey | A11057, Invitrogen | 1:500 |
| anti-rabbit | | |
| Cy™3 Donkey Anti-Goat | 705-165-147, Jackson | 1:500 |
| | Immuno Research | |
| Cy3-AffiniPure Donkey | 712-165-153, Jackson | 1:500 |
| anti-rat | Immuno Research | |
| Alexa Fluor 647 Donkey | 712-605-153, Jackson | 1:500 |
| Anti-Rat | Immuno Research | |
| Alexa Fluor 647 Donkey | 711-605-152, Jackson | 1:500 |
| anti-rabbit | Immuno Research | |
| Alexa Fluor 647 Donkey | 715-605-151, Jackson | 1:500 |
| anti-mouse | Immuno Research | |
| Alexa Fluor 647 Goat anti- | A-21451, Invitrogen | 1:500 |
| hamster | | |
| Biotin Donkey Anti-Rabbit | 711-065-152, Jackson | |
| | Immuno Research | |

For the p-SMAD2 staining experiment, 1X phosphatase inhibitor (Bimake, B15002) was added in 4% PFA during the tissue fixation process. The tyramide signal amplification method was used for pSMAD2 staining.

The human lung tissues were fixed with 4% PFA for 24 hours at 4° C., cryoprotected in 30% sucrose and embedded in OCT. All experiments were performed with the Institutional Review Board approval at both National Institute of Biological Sciences, Beijing, and China-Japan Friendship Hospital, Beijing.

Statistical analysis. All data are presented as mean±s.e.m. (as indicated in figure legends). The data presented in the figures were collected from multiple independent experiments that were performed on different days using different mice. Unless otherwise mentioned, most of the data presented in figure panels are based on at least three independent experiments. The inferential statistical significance of differences between sample means was evaluated using two-tailed unpaired Student's t-tests.

Isolating Mouse AT2 Cells

After 4 doses of tamoxifen injection, the lungs of Spc-CreER, Rosa26-mTmG mice were dissociated as previously described²³. Briefly, anesthetized mice were inflated with neutral protease (Worthington-Biochem, LS02111) and DNase I (Roche, 10104159001). AT2 cells were directly sorted based on the GFP fluorescence using the single-cell-select-mode in BD FACS Aria II and III appliances.

Quantitative RT-PCR (qPCR)

Total RNA was isolated from either whole lung or primary AT2 cells using Zymo Research RNA Mini Prep Kits (R2050). Reverse transcription reactions were performed with a two-step cDNA synthesis Kit (Takara, Cat. #6210A/B) according to the manufacturer's recommendations. qPCR was done with a CFX96 Touch™ Real-Time PCR Detection System. The mRNA levels of target genes were normalized to the Gapdh mRNA level. Primers used for qPCR are listed below.

Primers used for qPCR are listed below.

| | Forward | Reverse |
|---|---|---|
| Gapdh | AAGGTCGGTGTGAACGGAT | CGTTGAATTTGCCGTGAGT |
| | TTGG(SEQ ID NO: 8) | GGAG(SEQ ID NO: 9) |
| Areg | GCAGATACATCGAGAACCT | CCTTGTCATCCTCGCTGTG |
| | GGAG(SEQ ID NO: 10) | AGT(SEQ ID NO: 11) |
| Col1a1 | CCTCAGGGTATTGCTGGAC | CAGAAGGACCTTGTTTGCC |
| | AAC(SEQ ID NO: 12) | AGG(SEQ ID NO: 13) |

Areg Elisa

The mouse AREG immunoassay kit (R&D Systems, DY989) was used to detect the AREG concentration of the whole lung lysates. Specifically, the whole lung lobes were grinded in liquid nitrogen, then lysed using the cell lysis buffer. Then the lung lysates were added into the microplate wells applied. After the reaction, the absorbance at 450 nm was measured. The human areg immunoassay kit (abnova, B0RB01090J00018) was used to detect the AREG concentration of the human lung tissue lysates. Briefly, the human lung tissues were grinded in liquid nitrogen, then lysed using the cell lysis buffer. Then the lung lysates were added into the microplate wells applied. After the reaction, the absorbance at 450nm was measured. All experiments were performed with the Institutional Review Board approval at both National Institute of Biological Sciences, Beijing, and China-Japan Friendship Hospital, Beijing.

Primer sequence for sequencing the fragment of Cdc42 DNA sequence before and after deleting the exon2 of the Cdc42: Forward: CTGCCAACCATGACAACCTAA(SEQ ID NO:1); Reverse: AGACAAAACAACAAGGTCCAG (SEQ ID NO:2).

Primer sequences for sequencing the fragment of Areg DNA sequence before and after deleting the exon3 of the Areg: Forward: AAACAAAACAAGCTGAAATGTGG (SEQ ID NO: 4); Reverse: AAGGCCTTTAAGAACAAGTTGT (SEQ ID NO:15).

### Example 1. Generation and Characterization of Cdc42 AT2 Null Mice

In order to construct a progressive lung fibrosis animal model, Cdc42 AT2 null mice are generated by knocking out Cdc42 gene specifically in alveolar type II cells (AT2).

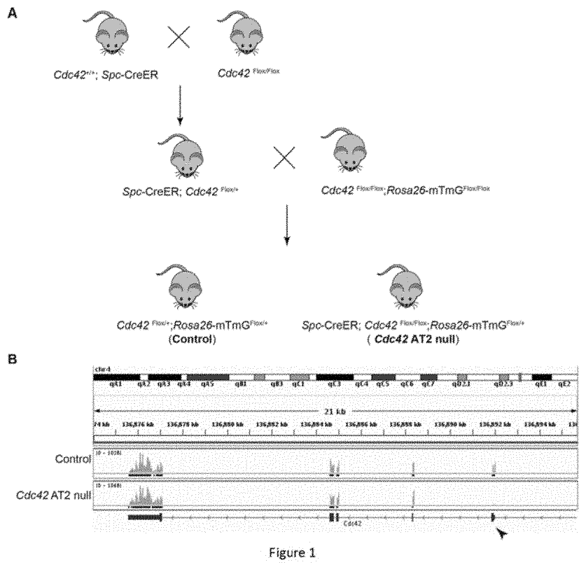

In order to specifically delete Cdc42 gene in AT2 cells, the mice carrying a Spc-CreER allele are crossed with the Cdc42 foxed (Cdc42^{flox/flox}) mice (FIG. 1A). In Cdc42^{flox/flox} mice, the exon 2 of Cdc42 gene, which contains the translation initiation exon of Cdc42 gene, is flanked by two loxp sites. In Spc-CreER; Cdc42^{flox/flox} mice, exon 2 of Cdc42 gene is specifically deleted in AT2 cells by Cre/loxp-mediated recombination after tamoxifen treatment (FIG. 1B). Spc-CreER; Cdc42^{flox/flox} mice are named as Cdc42 AT2 null mice.

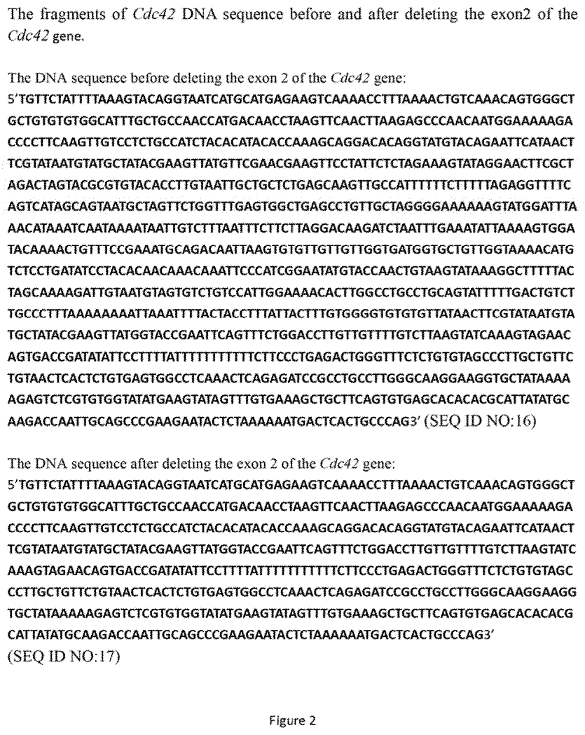

The fragments of Cdc42 DNA sequence before or after deleting the exon2 of the Cdc42 gene are shown in FIG. 2.

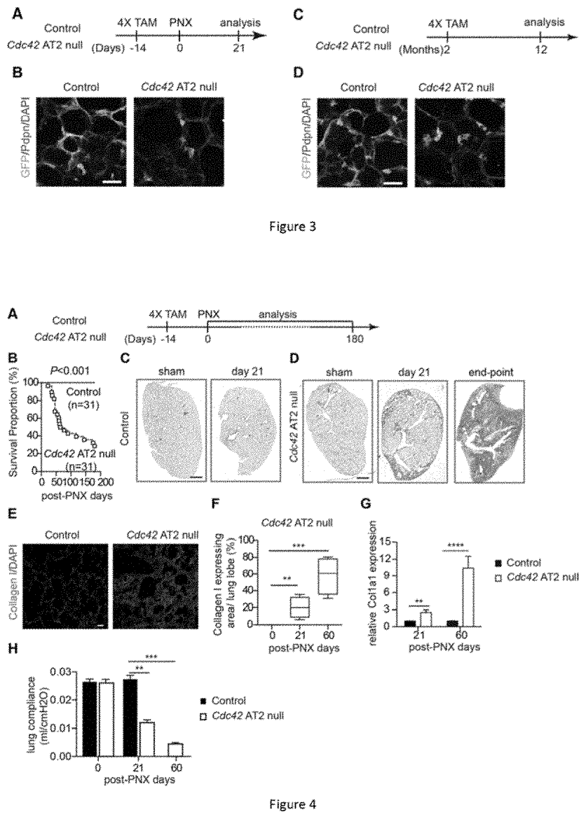

We performed PNX on control and Cdc42 AT2 null mice and analyzed the alveolar regeneration and AT2 cell differentiation at post-PNX day 21 (FIG. 3A). As shown in FIG. 3A, 200 μm lung sections of Control and Cdc42 AT2 null mice are immunostained with antibodies against GFP, Pdpn, and Prospc. At post-PNX day 21, many newly differentiated AT1 cells and newly formed alveoli are observed in no-prosthesis-implanted Control lungs (FIG. 3B). However, in Cdc42 AT2 null lungs, few AT2 cells have differentiated into AT1 cells, and no new alveoli are formed at post-PNX day 21 (FIG. 3B). It is observed that the alveoli in peripheral region of the Cdc42 AT2 null lungs are profoundly overstretched (FIG. 3B).

Under normal homeostatic conditions, AT2 cells slowly self-renew and differentiate into AT1 cells to establish new alveoli. To examine whether Cdc42 is required for AT2 cell differentiation during homeostasis, we deleted Cdc42 gene in AT2 cells when the mice were two-months old and analyzed the fate of AT2 cells until the mice were 12-month old. Lungs of Control and Cdc42 null mice without PNX treatment were collected at 12 months (FIG. 3C). Images show the maximum intensity of a 200 μm Z-projection of lung sections that were stained with antibodies against GFP, Pdpn, and Prospc. In the lungs of 12-month Control mice, we observed formation of many new alveoli (FIG. 3D). However, in the lungs of 12-month Cdc42 null mice (that had not undergone PNX), we observed enlarged alveoli with lacking any new AT1 cell formation (FIG. 3D).

Cdc42 AT2 null and Control mice after PNX are observed for a longer period of time (FIG. 4A). Surprisingly, some Cdc42 AT2 null mice show significant weight loss and increased respiration rates after post-PNX day 21. Indeed, fully 50% of PNX-treated Cdc42 AT2 null mice reach the predefined health-status criteria for endpoint euthanization by post-PNX day 60 (FIG. 4B), and about 80% of PNX-treated Cdc42 AT2 null mice reach their endpoints by post-PNX day 180 (FIG. 4B).

H&E staining of post-PNX Control and Cdc42 AT2 null mice reveals severe fibrosis in the lungs of Cdc42 AT2 null mice at their endpoints (FIG. 4D compared with FIG. 4C). In order to determine the point at which Cdc42 AT2 null mice begin to develop lung fibrosis following PNX, the lungs of Cdc42 AT2 null mice are analyzed at various time points after PNX using H&E staining (FIG. 4D). The subpleural regions of some Cdc42 AT2 null lungs exhibit signs of tissue thickening by post-PNX day 21 (FIG. 4D). By the end-point, the dense fibrosis has progressed to the center of most Cdc42 AT2 null lungs (FIG. 5D). What we have observed in post-PNX and aged Cdc42 null mice is similar to the characteristic progression of IPF, in which fibrotic lesions first occur at the lung periphery and subsequently progress inward towards the center of lung lobes.

In addition to detecting strong immunofluorescence signals for Collagen I in these dense fibrotic regions of lungs of Cdc42 AT2 null mice (FIG. 4E), we observe the proportion of Collagen I expressing area per lobe gradually increased after PNX in Cdc42 AT2 null mice (FIG. 4F). Our qPCR analysis also shows that the Collagen I mRNA expression levels increased gradually from post-PNX day 21 (FIG. 4G). Moreover, gradually decreased lung compliance is observed in PNX-treated Cdc42 AT2 null mice from post-PNX day 21 as compared to their PNX-treated Control mice (FIG. 4H), an intriguing finding given that decreased lung compliance is known to occur frequently as lungs become fibrotic²³.

Since it is found that impaired AT2 differentiation and enlarged alveoli in 12-month old Cdc42 AT2 null mice (FIG. 3D), then lungs of control and Cdc42 AT2 null mice without PNX treatment are analyzed from 10-months of age to 24-months of age (FIG. 5A). Fibrotic changes in the lungs of control mice are never observed, even the control mice reached 24-months of age (FIG. 5B). We found no significant fibrotic changes before the Cdc42 AT2 null mice reached 10-months of age (FIG. 5C). It is also observed that by 12 months, fibrosis has obviously begun to develop in the subpleural regions of Cdc42 AT2 null lungs and to progress toward the center of the lung after 12 months (FIG. 5C).

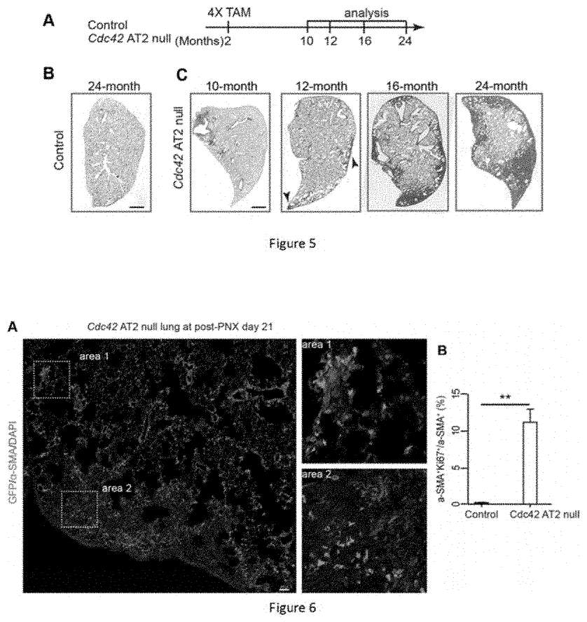

Fibroblastic foci are considered as a relevant morphologic marker of progressive pulmonary fibrosis and are recognized as sites where fibrotic responses are initiated and/or perpetuated in progressive pulmonary fibrosis³⁵. The fibroblastic foci contain proliferating α-SMA⁺ fibroblasts. Lungs of Cdc42 AT2 null mice at post-PNX day 21 are stained with antibodies against α-SMA (FIG. 6A). Some α-SMA⁺ fibroblasts started to accumulate next to a cluster of AT2 cells in the relative normal alveolar regions of Cdc42 AT2 null lungs are observed (area 1, FIG. 6A). And the dense fibrosis region of the lungs is filled with α-SMA⁺ fibroblasts (area 2, FIG. 6A). In addition, by immunostaining using antibodies against both α-SMA and proliferation marker, Ki67, we show that the cell proliferation of α-SMA⁺ cells is increased dramatically in the lungs of Cdc42 AT2 null mice at post-PNX day 21. These results indicate that the proliferating α-SMA⁺ fibroblasts contribute to the development of lung fibrosis of Cdc42 AT2 null mice (FIG. 6B).

Collectively, the loss of Cdc42 in AT2 cells leads to progressive lung fibrosis in PNX-treated mice. Moreover, this progressive lung fibrosis phenotype also occurs in no-PNX-treated Cdc42 AT2 null mice starting from around 12 months of age. All these results demonstrate that deletion of Cdc42 in AT2 cells leads to IPF like progressive pulmonary fibrosis in mice, and therefore, a mouse model of IPF like progressive lung fibrosis is established and can be used to study human IPF disease.

### Example 2. Sequence Characterization of the Cdc42 AT2 Null Mice

The Spc-CreER, Cdc42^{flox/-} mice were performed genome purification and PCR amplification. Then the fox and null bands of Cdc42 were purified and sequenced using the primers as below: CTGCCAACCATGACAACCTAA (SEQ ID NO.1): AGACAAAACAACAAGGTCCAG (SEQ ID NO:2).

The fragments of Cdc42 DNA sequence before or after deleting the exon2 of the Cdc42 gene are shown in FIG. 2.

### Example 3. Amphiregulin (AREG) is Strongly Expressed in AT2 Cells of Cdc42 AT2 Null Lungs After PNX Treatment

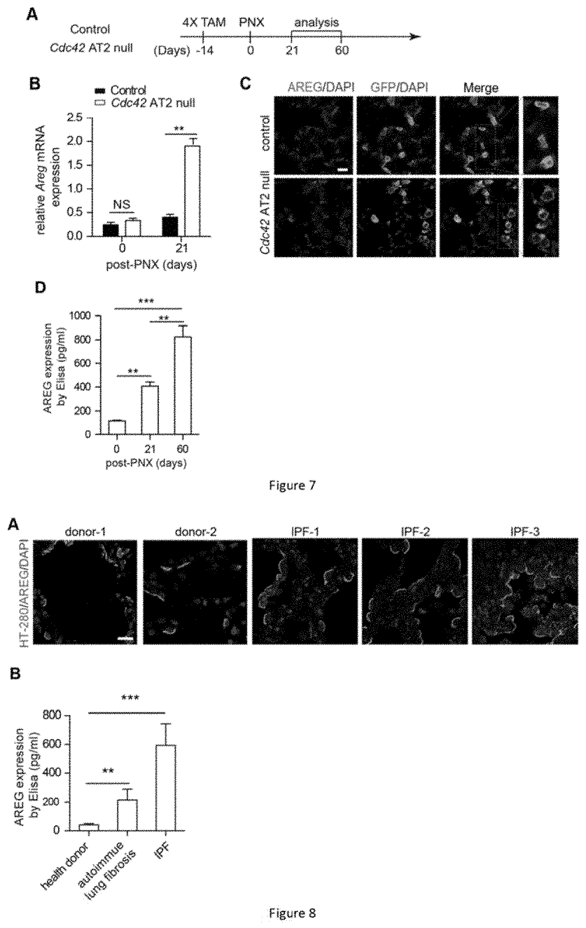

In the Cdc42 AT2 null fibrosis model, the Cdc42 AT2 null lungs start to show fibrotic changes at post-PNX day 21 (FIG. 4D). We have characterized the Control and Cdc42 null AT2 cells after PNX treatment (FIG. 7A). It is observed that Areg is one of the most upregulated genes in AT2 cells of Cdc42 AT2 null lungs at post-PNX day 21 by both RNA sequencing analysis and quantitative PCR (qPCR) (FIG. 7B). By immunostaining, it is observed that AREG can be detected in AT2 cells of Cdc42 AT2 null lungs at post-PNX day 21 (FIG. 7C). No AREG signal can be detected in control lungs at post-PNX day 21 (FIG. 7C), which is consistent with the information from the human tissue atlas that the expression of AREG is under the detectable level in adult lung tissues. In addition, the AREG signal is specifically detected in AT2 cells. The expression of AREG protein in Cdc42 AT2 null lungs is measured by an AREG Elisa kit. It is observed that the expression levels of AREG are gradually increased from post-PNX day 21 to post-PNX day 60 in the lungs of Cdc42 AT2 null mice (FIG. 7D).

### Example 4. AREG is Strongly Expressed in AT2 Cells of Pulmonary Fibrosis Patients

As shown in Example 3, the positive correlation between the expression level of AREG and the progression of lung fibrosis in Cdc42 AT2 null mice is observed. The expression levels of AREG in 2 donor and 3 IPF lungs are analyzed. Remarkably, it is observed that AREG is detected in AT2 cells (HTII-280 expressing cells) of all IPF specimens but is not detected in AT2 cells of donor lungs (FIG. 8A). The expression of AREG in lungs of IPF patients and patients with autoimmune induced lung fibrosis is measured by an AREG Elisa kit. It is found that the expression levels of AREG are significantly increased in the lungs of IPF patients and patients with autoimmune induced lung fibrosis (FIG. 8B).

Together, these results show that the expression level of AREG is significantly up-regulated in AT2 cells of the both progressive fibrosis mouse model and lung fibrosis patients.

### Example 5. Overexpressing AREG in AT2 Cells is Sufficiently to Induce Lung Fibrosis

Generation of Teto-Areg Mice

Insert a tetracycline response element before CMV promoter-driven Areg so that the expression of Areg can induced when mice are treated with doxycycline (Dox). The sequence of tetracycline response element is shown as followed:

(SEQ ID NO: 5)
5′TCCCTATCAGTGATAGAGAAAAGTGAAAGTCGAGTTTACCACTCCCTA
TCAGTGATAGAGAAAAGTGAAAGTCGAGTTTACCACTCCCTATCAGTGAT
AGAGAAAAGTGAAAGTCGAGTTTACCACTCCCTATCAGTGATAGAGAAAA
GTGAAAGTCGAGTTTACCACTCCCTATCAGTGATAGAGAAAAGTGAAAGT
CGAGTTTACCACTCCCTATCAGTGATAGAGAAAAGTGAAAGTCGAGTTTA
CCACTCCCTATCAGTGATAGAGA3′.

Insert a minimal CMV promoter before Areg cDNA so that Areg is overexpressed. The sequence of CMV promter is shown as followed:

5

(SEQ ID NO: 6)
5′GGTAGGCGTGTACGGTGGGAGGCCTATATAAGCAGAGCT3′.

The sequence of Areg cDNA is shown as followed:

(SEQ ID NO: 7)
5′ATGAGAACTCCGCTGCTACCGCTGGCGCGCTCAGTGCTGTTGCTGCTG
GTCTTAGGCTCAGGCCATTATGCAGCTGCTTTGGAGCTCAATGACCCCAG
CTCAGGGAAAGGCGAATCGCTTTCTGGGGACCACAGTGCCGGTGGACTTG
AGCTTTCTGTGGGAAGAGAGGTTTCCACCATAAGCGAAATGCCTTCTGGC
AGTGAACTCTCCACAGGGGACTACGACTACTCAGAGGAGTATGATAATGA
ACCACAAATATCCGGCTATATTATAGATGATTCAGTCAGAGTTGAACAGG
TGATTAAGCCCAAGAAAAACAAGACAGAAGGAGAAAAGTCTACAGAAAAA
CCCAAAAGGAAGAAAAAGGGAGGCAAAAATGGAAAAGGCAGAAGGAATAA
GAAGAAAAAGAATCCATGCACTGCCAAGTTTCAGAACTTTTGCATTCATG
GCGAATGCAGATACATCGAGAACCTGGAGGTGGTGACATGCAATTGTCAT
CAAGATTACTTTGGTGAACGGTGTGGAGAAAAATCCATGAAGACTCACAG
CGAGGATGACAAGGACCTATCCAAGATTGCAGTAGTAGCTGTCACTATCT
TTGTCTCTGCCATCATCCTCGCAGCTATTGGCATCGGCATCGTTATCACA
GTGCACCTTTGGAAACGATACTTCAGGGAATATGAAGGAGAAACAGAAGA
AAGAAGGAGGCTTCGACAAGAAAACGGGACTGTGCATGCCATTGCCTAG
3′.

The tetracycline response element, CMV promoter, and Areg CDNA were enzyme-linked and inserted into the mouse genome. The sequence of teto-Areg is shown as followed:

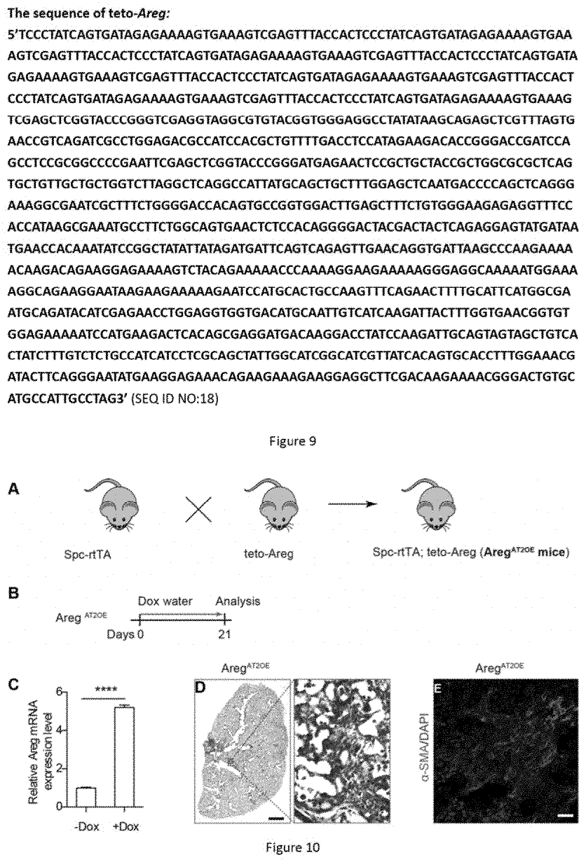

(SEQ ID NO: 18)
5′TCCCTATCAGTGATAGAGAAAAGTGAAAGTCGAGTTTACCACTCCCTA
TCAGTGATAGAGAAAAGTGAAAGTCGAGTTTACCACTCCCTATCAGTGAT
AGAGAAAAGTGAAAGTCGAGTTTACCACTCCCTATCAGTGATAGAGAAAA
GTGAAAGTCGAGTTTACCACTCCCTATCAGTGATAGAGAAAAGTGAAAGT
CGAGTTTACCACTCCCTATCAGTGATAGAGAAAAGTGAAAGTCGAGTTTA
CCACTCCCTATCAGTGATAGAGAAAAGTGAAAGTCGAGCTCGGTACCCGG
GTCGAGGTAGGCGTGTACGGTGGGAGGCCTATATAAGCAGAGCTCGTTTA
GTGAACCGTCAGATCGCCTGGAGACGCCATCCACGCTGTTTTGACCTCCA
TAGAAGACACCGGGACCGATCCAGCCTCCGCGGCCCCGAATTCGAGCTCG
GTACCCGGGATGAGAACTCCGCTGCTACCGCTGGCGCGCTCAGTGCTGTT
GCTGCTGGTCTTAGGCTCAGGCCATTATGCAGCTGCTTTGGAGCTCAATG
ACCCCAGCTCAGGGAAAGGCGAATCGCTTTCTGGGGACCACAGTGCCGGT
GGACTTGAGCTTTCTGTGGGAAGAGAGGTTTCCACCATAAGCGAAATGCC
TTCTGGCAGTGAACTCTCCACAGGGGACTACGACTACTCAGAGGAGTATG
ATAATGAACCACAAATATCCGGCTATATTATAGATGATTCAGTCAGAGTT
GAACAGGTGATTAAGCCCAAGAAAAACAAGACAGAAGGAGAAAAGTCTAC
AGAAAAACCCAAAAGGAAGAAAAAGGGAGGCAAAAATGGAAAAGGCAGAA
GGAATAAGAAGAAAAAGAATCCATGCACTGCCAAGTTTCAGAACTTTTGC
ATTCATGGCGAATGCAGATACATCGAGAACCTGGAGGTGGTGACATGCAA
TTGTCATCAAGATTACTTTGGTGAACGGTGTGGAGAAAAATCCATGAAGA
CTCACAGCGAGGATGACAAGGACCTATCCAAGATTGCAGTAGTAGCTGTC
ACTATCTTTGTCTCTGCCATCATCCTCGCAGCTATTGGCATCGGCATCGT
TATCACAGTGCACCTTTGGAAACGATACTTCAGGGAATATGAAGGAGAAA
CAGAAGAAAGAAGGAGGCTTCGACAAGAAAACGGGACTGTGCATGCCATT
GCCTAG3′(FIG. 9).

In Spc-rtTA; teto-Areg mice, the expression of Areg was induced specifically in AT2 cells after the doxycycline treatment.

Primer sequences for sequencing Leto-Areg sequence are shown as follo red:

Forward:
(SEQ ID NO: 19)
GTACCCGGGATGAGAACTCCG;
Reverse:
(SEQ ID NO: 20)
GCCGGATATTTGTGGTTCATT.

In order to assess the function of increased expression of AREG in AT2 cells, Areg AT2 overexpression transgenic mice, in which Areg can be specifically overexpressed in AT2 cells, are generated. Firstly, transgenic mice that express Areg under the control of a tetracycline-responsive promoter element (tetO) are generated. The mice that carry the allele of Spc-rtTA are crossed with mice that carry the allele of teto-Areg in order to get the offspring mice that carry Spc-rtTA; teto-Areg. When exposing the Spc-rtTA; teto-Areg mice to the tetracycline analog, doxycycline (Dox), the expression of Areg is specifically induced in AT2 cells. The Spc-rtTA; teto-Areg mice are named as Areg^{AT2OE} mice (FIG. 10A).

The Areg^{AT2OE} mice are treated with Dox-containing water for 21 days (FIG. 10B). Then the lungs of Areg^{AT2OE} mice with or without Dox treatment are collected for analysis. qPCR analysis shows that the expression of Areg mRNA is significantly induced in AT2 cells of Areg^{AT2OE} mice after the Dox treatment (FIG. 10C). H&E staining shows that lungs of Dox-treated Areg^{AT2OE} mice have obvious fibrotic changes (FIG. 10D). Many cells in fibrotic region express high levels of α-SMA (FIG. 10E).

For the first time, these results indicate that ectopic expression of AREG in AT2 cells is sufficient to induce pulmonary fibrosis.

### Example 6. Generation of Areg AT2 Null Mice

Generating Areg^{flox/flox} mice: the Areg^{flox/flox} mice were generated according to the previous work³³. Briefly, the Areg exon3 was anchored by loxp. The loxpl (GACACGGA TCCATAACTTCGTATAATGTATGCTATACGAAGTTATCGAGTC (SEQ ID NO:3)) was inserted into the Areg DNA position 3704, and the loxp2 (CCGCGGATAACTTC GTATAATGTATGCTATACGAAGTTATACTAGTCCAACG(SEQ ID NO:4)) was inserted into the Areg DNA position 4208. After the tamoxifen-induced Cre-loxP recombination, the Areg exon3 was deleted then the AREG function was blocked.

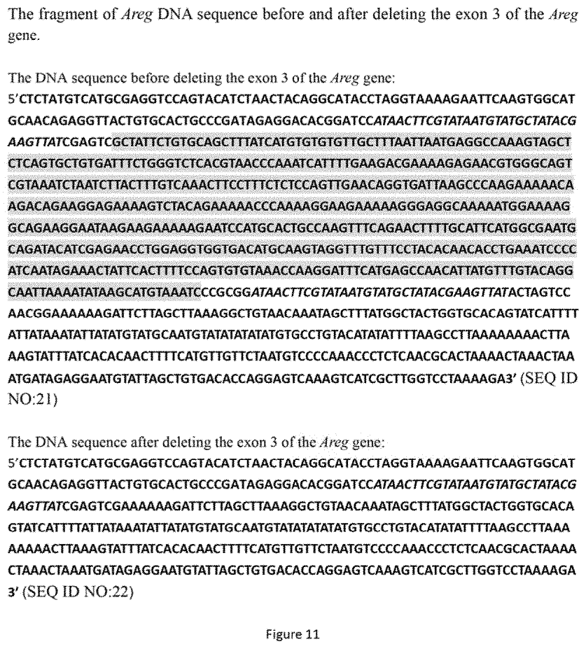

The fragments of Areg DNA sequence before or after deleting the exon3 of the Areg gene are shown in FIG. 11.

### Example 7. Deleting Areg Gene in Cdc42 Null AT2 Cells Significantly Attenuated the Development of Lung Fibrosis

Given the fibrotic function of AREG in AT2 cells, whether reducing the expression level of AREG in Cdc42 null AT2 cells will attenuate the fibrosis development in Cdc42 AT2 null lungs is assessed. Areg flox mice in which the exons 3 of Areg gene are flanked by two loxp sites are generated. The mice, in which Areg gene was deleted in whole body, are analyzed. The Areg^{-/-} mice are viable and fertile, suggesting that Areg gene is not essential for the survival and development of mice. After several generations of crossings, we obtain Areg&Cdc42 AT2 double null mice, in which Areg and Cdc42 genes are both deleted in AT2 cells.

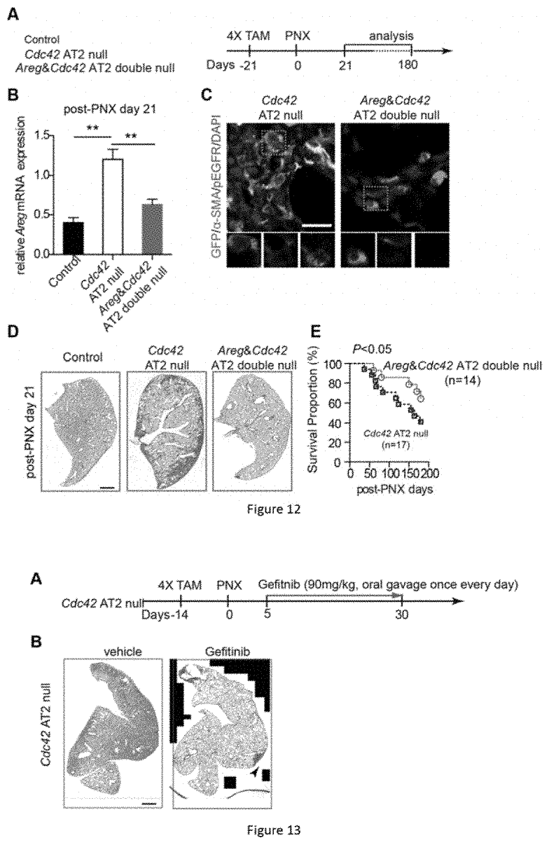

Thereafter, the effect of deleting Areg genes in Cdc42 null AT2 cells is investigated. Control, Cdc42 AT2 null, and Areg&Cdc42 AT2 double null mice are exposed to 4 doses of tamoxifen 14 days prior to PNX (FIG. 12A). Lungs of these mice are analyzed at the various time points post-PNX. At post-PNX day 21, qPCR analysis has shown that the expression level of Areg in Areg&Cdc42 double null AT2 cells is not increased at post-PNX day 21, demonstrating the deletion of Areg gene in the AT2 cells (FIG. 12B).

AREG binds to EGFR, which can activate the phosphorylation of EGFR. The p-EGFR expression in α-SMA⁺ fibroblasts is examined by an immunostaining experiment using an antibody against GFP (labeling AT2 cells), p-EGFR, and α-SMA. Strong p-EGFR expression in α-SMA positive fibroblasts in Cdc42 AT2 null lungs is observed (FIG. 12C). In Areg&Cdc42 AT2 double null lungs, not only much less α-SMA positive fibroblasts is detected, but also decreased expression level of p-EGFR (FIG. 12C) is observed. This demonstrates that the strength of EGFR signaling in α-SMA positive fibroblasts is dependent on the AREG expression in AT2 cells. In addition, Areg&Cdc42 AT2 double null lungs show minimal fibrosis at post-PNX day 21, as compared to the significant lung fibrosis in Cdc42 AT2 null lungs (FIG. 12D). The survival curve also shows that Areg&Cdc42 AT2 double null mice have a significant prolongation of lifespan compared to Cdc42 AT2 null mice (FIG. 12E).

Together, these results demonstrate that reducing the expression level of AREG in AT2 cells significantly attenuated the development of pulmonary fibrosis of Cdc42 AT2 null mice. These results also indicate that AREG and its receptor, EGFR, are therapeutic targets for treating fibrosis.

### Example 8. Sequence Characterization of the Areg AT2 Null Mice

The Spc-CreER, Areg^{flox/-} mice were performed genome purification and PCR amplification. Then the fox and null bands of Areg were purified and sequenced using the primers as below: AAACAAAACAAGCTGAAATGTGG (SEQ ID NO:14); AAGGCCTTTAAGAACAAGTTGT (SEQ ID NO:15).

### Example 9. Targeting AREG and its Receptor, EGFR, to Treat IPF and Other Fibrosis Diseases

Given the fact that EGFR in α-SMA positive fibroblasts can be activated by AREG (FIG. 12C), the effect of inhibiting the activity of AREG receptor, EGFR, on the progression of lung fibrosis is investigated. PNX-treated Cdc42 AT2 null mice are treated with PBS only, or are treated with an inhibitor of EGFR, Gefitnib, from post-PNX day 6 to post-PNX day 30 (FIG. 13A). It is found that Gefitnib treatment also significantly inhibits the fibrosis development in the lungs of Cdc42 AT2 null mice (FIG. 13B).

Taking together, these results demonstrate that blocking AREG and its receptor, EGFR, is an ideal therapeutic approach for treating the IPF and other fibrosis diseases.

### REFERENCE

1 Wynn, T. A. Cellular and molecular mechanisms of fibrosis. The Journal of pathology 214, 199-210, doi:10.1002/path.2277 (2008).

2 Wynn, T. A. & Ramalingam, T. R. Mechanisms of fibrosis: therapeutic translation for fibrotic disease. Nature medicine 18, 1028-1040, doi:10.1038/nm.2807 (2012).

3 Mehal, W. Z., Iredale, J. & Friedman, S. L. Scraping fibrosis: expressway to the core of fibrosis. Nature medicine 17, 552-553, doi:10.1038/nm0511-552 (2011).

4 Barkauskas, C. E. & Noble, P. W. Cellular mechanisms of tissue fibrosis. 7. New insights into the cellular mechanisms of pulmonary fibrosis. American journal of physiology. Cell physiology 306, C987-996, doi:10.1152/ajpce11.00321.2013 (2014).

5 Rock, J. R. et al. Multiple stromal populations contribute to pulmonary fibrosis without evidence for epithelial to mesenchymal transition. Proceedings of the National Academy of Sciences of the United States of America 108, E1475-1483, doi:10.1073/pnas.1117988108 (2011).

6 Gross, T. J. & Hunninghake, G. W. Idiopathic pulmonary fibrosis. New England Journal of Medicine 345, 517-525 (2001).

7 Vyalov, S. L., Gabbiani, G. & Kapanci, Y. Rat alveolar myofibroblasts acquire alpha-smooth muscle actin expression during bleomycin-induced pulmonary fibrosis. The American journal of pathology 143, 1754 (1993).

8 King Jr, T. E., Pardo, A. & Selman, M. Idiopathic pulmonary fibrosis. The Lancet 378, 1949-1961 (2011).

9 Plantier, L. et al. Ectopic respiratory epithelial cell differentiation in bronchiolised distal airspaces in idiopathic pulmonary fibrosis. Thorax 66, 651-657, doi:10.1136/thx.2010.151555 (2011).

10 Steele, M. P. & Schwartz, D. A. Molecular mechanisms in progressive idiopathic pulmonary fibrosis. Annual review of medicine 64, 265-276, doi:10.1146/annurev-med-042711-142004 (2013).

11 Camelo, A., Dunmore, R., Sleeman, M. A. & Clarke, D. L. The epithelium in idiopathic pulmonary fibrosis: breaking the barrier. Frontiers in pharmacology 4, 173, doi:10.3389/fphar.2013.00173 (2014).

12 Barkauskas, C. E. et al. Type 2 alveolar cells are stem cells in adult lung. The Journal of clinical investigation 123, 3025-3036, doi:10.1172/JCI68782 (2013).

13 Desai, T. J., Brownfield, D. G. & Krasnow, M. A. Alveolar progenitor and stem cells in lung development, renewal and cancer. Nature 507, 190-194, doi:10.1038/nature12930 (2014).

14 Haies, D. M., Gil, J. & Weibel, E. R. Morphometric study of rat lung cells: I. Numerical and dimensional characteristics of parenchymal cell population. American Review of Respiratory Disease 123, 533-541 (1981).

15 Selman, M. & Pardo, A. Idiopathic pulmonary fibrosis: an epithelial/fibroblastic cross-talk disorder. Respiratory research 3, 3 (2001).

16 Kropski, J. A., Blackwell, T. S. & Loyd, J. E. The genetic basis of idiopathic pulmonary fibrosis. European Respiratory Journal 45, 1717-1727 (2015).

17 Goodwin, A. T. & Jenkins, G. Molecular endotyping of pulmonary fibrosis. Chest 149, 228-237 (2016).

18 Xu, Y. et al. Single-cell RNA sequencing identifies diverse roles of epithelial cells in idiopathic pulmonary fibrosis. JCI insight 1 (2016).

19 Sternlicht, M. D. & Sunnarborg, S. W. The ADAM17—amphiregulin—EGFR axis in mammary development and cancer. Journal of mammary gland biology and neoplasia 13, 181-194 (2008).

20 Berasain, C. & Avila, M. A. in Seminars in cell & developmental biology. 31-41 (Elsevier).

21 Sternlicht, M. D. et al. Mammary ductal morphogenesis requires paracrine activation of stromal EGFR via ADAM17-dependent shedding of epithelial amphiregulin. Development 132, 3923-3933 (2005).

22 Macias, H. & Hinck, L. Mammary gland development. Wiley Interdisciplinary Reviews:

Developmental Biology 1, 533-557 (2012).

23 (!!! INVALID CITATION !!!).

24 Busser, B., Sancey, L., Brambilla, E., Coll, J.-L. & Hurbin, A. The multiple roles of amphiregulin in human cancer. Biochimica et Biophysica Acta (BBA)-Reviews on Cancer 1816, 119-131 (2011).

25 Chen, Z. et al. Aberrantly activated AREG—EGFR signaling is required for the growth and survival of CRTC1—MAML2 fusion-positive mucoepidermoid carcinoma cells. Oncogene 33, 3869 (2014).

26 Busser, B. et al. Amphiregulin promotes resistance to gefitinib in nonsmall cell lung cancer cells by regulating Ku70 acetylation. Molecular Therapy 18, 536-543 (2010).

27 Wang, X., Masri, S., Phung, S. & Chen, S. The role of amphiregulin in exemestane-resistant breast cancer cells: evidence of an autocrine loop. Cancer research 68, 2259-2265 (2008).

28 Zhou, Y. et al. Amphiregulin, an epidermal growth factor receptor ligand, plays an essential role in the pathogenesis of transforming growth factor-f3-induced pulmonary fibrosis. Journal of Biological Chemistry 287, 41991-42000 (2012).

29 Steele, M. P. & Schwartz, D. A. Molecular mechanisms in progressive idiopathic pulmonary fibrosis. Annual review of medicine 64, 265-276 (2013).

30 Chen, L. et al. Cdc42 deficiency causes Sonic hedgehog-independent holoprosencephaly. Proceedings of the National Academy of Sciences 103, 16520-16525 (2006).

31 Council, N. R. Guide for the care and use of laboratory animals. (National Academies

Press, 2010).

32 Foltz, C. J. & Ullman-Cullere, M. Guidelines for assessing the health and condition of mice. Resource 28 (1999).

33 Luetteke, N. C. et al. Targeted inactivation of the EGF and amphiregulin genes reveals distinct roles for EGF receptor ligands in mouse mammary gland development. Development 126, 2739-2750 (1999).

34 Wang, Y. et al. Pulmonary alveolar type I cell population consists of two distinct subtypes that differ in cell fate. Proceedings of the National Academy of Sciences, 201719474 (2018).

35 Lynch, D. A. et al. Diagnostic criteria for idiopathic pulmonary fibrosis: a Fleischner Society White Paper. The Lancet Respiratory Medicine 6, 138-153, doi:10.1016/s2213-2600(17)30433-2 (2018).

1. A drug target for idiopathic pulmonary fibrosis (IPF), which is AREG signaling in AT2 cells of lung from an animal or a human being.

2. The drug target of claim 1, wherein AREG is detected in AT2 cells of lung from animals and human beings, suffering from idiopathic pulmonary fibrosis, and is absent in AT2 cells of normal lung from an animal or a human being.

3. The drug target of claim 1, wherein AREG is detected in AT2 cells of Cdc42 AT2 null lung, and the expression level of AREG is increased in AT2 cells of Cdc42 AT2 null lung after PNX.

4. The drug target of claim 1, wherein the expression level of AREG is up-regulated in AT2 cells of lung from an animal or a human being suffering from progressive fibrosis.

5. The drug target of claim 1, wherein the AREG signaling in AT2 cells of lung from an animal or a human being is AREG target.

6. The drug target of claim 5, wherein the AREG target is AREG in AT2 cells of lung from an animal or a human being.

7. (canceled).

8. The drug target of claim 5, wherein the AREG target is EGFR in fibroblasts of lung from an animal or a human being.

9. The drug target of claim 8, wherein the strength of EGFR signaling in α-SMA positive fibroblasts is dependent on the AREG expression in AT2 cells.

10. The drug target of claim 1, wherein the drug target is inhibited to reduce the expression level of AREG in AT2 cells of lung from an animal or a human being.

11-15. (Canceled).

16. A transgenic mouse, wherein AREG is specifically overexpressed in AT2 cells of lungs.

17. (canceled).

18. The transgenic mouse of claim 16, wherein the transgenic mouse is Spc-rtTA; teto-Areg mouse.

19. The transgenic mouse of claim 18, wherein the Spc-rtTA; teto-Areg mouse has a characterized sequence shown by SEQ ID NO:18.

20. The transgenic mouse of claim 19, wherein the Spc-rtTA; teto-Areg mouse can be identified using the following primer sequences:
Forward:
(SEQ ID NO: 19)
GTACCCGGGATGAGAACTCCG;
Reverse:
(SEQ ID NO: 20)
GCCGGATATTTGTGGTTCATT.

21. (canceled) .

22. A method for screening a drug for treating pulmonary fibrosis of an animal or a human being, by using an AREG signaling in AT2 cells of lung front an animal or a human being as a drug target.

23. A method for diagnosing pulmonary fibrosis of an animal or a human being, comprising contacting a detector of AREG and/or a detector of its receptor EGFR with a sample from an animal or a human being suspected suffering from pulmonary fibrosis.

24. (Canceled).

25. The method of claim 23, wherein the sample is the biopsy tissue from the animals or the human being.

26. The mothod of claim 25, wherein AREG is detected in the biopsy tissue, and then the animals or the human being is diagnosed as suffering from a server pulmonary fibrosis.

27. A method for treating pulmonary fibrosis of an animal or a human being, comprising administering a subject with a therapeutically effective amount of a substance targeting AREG in AT2 cells and/or its receptor.

28. The method of claim 27, wherein the substance is an inhibitor of AREG in AT2 cells, or is an inhibitor of EGFR in fibroblasts of lungs.

29. The drug target of claim 1, wherein the animal is mouse, rabbit, rat, canine, pig, horse, cow, sheep, monkey or chimpanzee.

30. The method of claim 22, wherein the animal is mouse, rabbit, rat, canine, pig, horse, cow, sheep, monkey or chimpanzee.

31. The method of claim 23, wherein the animal is mouse, rabbit, rat, canine, pig, horse, cow, sheep, monkey or chimpanzee.

32. The method of claim 22, wherein the pulmonary fibrosis is idiopathic pulmonary fibrosis.

33. The method of claim 23, wherein the pulmonary fibrosis is idiopathic pulmonary fibrosis.

34. The method of claim 25, wherein the biopsy tissue is lung tissue from the animals or the human being.

35. The method of claim 25, wherein the biopsy tissue is the lower part, the middle part or the upper part of the lung lobe from the animals or the human being.

36. The method of claim 27, wherein the pulmonary fibrosis is idiopathic pulmonary fibrosis.

37. The method of claim 27, wherein the receptor is EGFR in fibroblasts of lungs.
